(19) United States
(12) Patent Application Publication
(10) Pub. No.: US 2022/0292737 A1
(43) Pub. Date: Sep. 15, 2022

(54) METHOD FOR CONVERTING MRI TO CT IMAGE BASED ON ARTIFICIAL INTELLIGENCE, AND ULTRASOUND TREATMENT DEVICE USING THE SAME

(71) Applicant: KOREA INSTITUTE OF SCIENCE AND TECHNOLOGY, Seoul (KR)

(72) Inventors: Hyung Min KIM, Seoul (KR); Kyungho YOON, Seoul (KR); Tae Young PARK, Seoul (KR); Heekyung KOH, Seoul (KR)

(73) Assignee: KOREA INSTITUTE OF SCIENCE AND TECHNOLOGY, Seoul (KR)

(21) Appl. No.: 17/689,032

(22) Filed: Mar. 8, 2022

(30) Foreign Application Priority Data
Mar. 11, 2021 (KR) 10-2021-0031884

(51) Int. Cl. G06T 11/00; G06T 7/11; A61N 7/02; A61B 5/00; A61B 5/055; G16H 30/40

(52) U.S. Cl. CPC G06T 11/005; G06T 7/11; A61N 7/02; A61B 5/0036; A61B 5/055; A61B 5/7267; A61B 5/7278; G16H 30/40; G06T 2207/20081; G06T 2207/10081; G06T 2207/10088; G06T 2207/20084; G06T 2207/20221; G06T 2207/20021; G06T 2207/30016; G06T 2210/41; A61N 2007/0026

(57) ABSTRACT

The present disclosure relates to a method for converting magnetic resonance imaging (MRI) to a computed tomography (CT) image using an artificial intelligence machine learning model, for use in ultrasound treatment device applications. The method includes acquiring training data including an MRI image and a CT image for machine learning; training an artificial neural network model using the training data, wherein artificial neural network model generates a CT image corresponding to the MRI image, and compares the generated CT image with the original CT image included in the training data; receiving an input MRI image to be converted to a CT image; splitting the input MRI image into a plurality of patches; generating patches of a CT image corresponding to the patches of the input MRI image using the trained artificial neural network model; and merging the patches of the CT image to generate an output CT image.

### CROSS-REFERENCE TO RELATED APPLICATION

This application claims priority to Korean Patent Application No. 10-2021-0031884, filed on Mar. 11, 2021, and all the benefits accruing therefrom under 35 U.S.C. § 119, the contents of which in its entirety are herein incorporated by reference.

### BACKGROUND OF THE INVENTION

### Field of the Invention

The present disclosure relates to a method for converting a magnetic resonance imaging (MRI) image to a computed tomography (CT) image using an artificial intelligence machine learning model and an ultrasound treatment device using the same.

### Description of Government-Funded Research and Development

This research is conducted by Korea Evaluation Institute of Industrial Technology under the support of Ministry of Trade, Industry and Energy (Project name: Development of technology for personalized B2B (Brain to Brain) cognitive enhancement based on high resolution noninvasive bidirectional neural interfaces, Project No.: 1415169864).

This research is conducted by National Research Council of Science & Technology under the support of Ministry of Science and ICT (Project name: Development of technology for overcoming barriers to stroke patients based on personalized neural plasticity assessment and enhancement, Project No.: CAP-18-01-KIST).

### Description of the Related Art

Conventionally, the insertion of electrodes into a patient's body has been used to conduct therapy for the patient's pain relief or stimulation of the nerve cell of the specific body part, but there is the risk of damage to the human body by the physically invasive process.

Recently, ultrasound stimulation therapy that stimulates an affected part without a physically invasive process is widely used, and ultrasound may be classified into High-intensity Focused Ultrasound (HIFU) and Low-intensity Focused Ultrasound (LIFU) according to the output intensity. The HIFU is used in direct treatment for thermally and mechanically removing living tissues such as cancer cells, tumors and lesions, while the LIFU is widely used to treat brain diseases such as Alzheimer's disease and depression by stimulating brain nerves or can be used in rehabilitation therapy to induce neuromuscular activation by stimulation. The focused ultrasound treatment technology is gaining attention due to its minimally invasive process with fewer side effects such as infection or complications.

Magnetic Resonance guided Focused Ultrasound (MRgFUS) treatment technology combines focused ultrasound treatment technology with image-guided technology. The image-guided surgery is chiefly used, for example, in a neurological surgery or an implant surgery in which it is difficult for a surgeon to directly see a patient's affected part and the surgeon is required to conduct the surgery while avoiding the major nerves and organs in the patient's body. In general, ultrasound treatment is performed while observing the surgical site in magnetic resonance imaging (MRI) images acquired through MRI equipment or computed tomography (CT) images acquired through CT equipment.

In particular, transcranial MRgFUS using ultrasonic energy delivered through the skull requires MRI as well as CT scans, and CT images are used to find skull factors and acoustic parameters necessary for proper penetration of ultrasonic energy. For example, acoustic property information essential for ultrasound treatment such as speed of sound, density and attenuation coefficient may be acquired using skull factor information identified through CT scans.

However, the addition of CT scans increases the temporal and economical burden of the patient and the medical staff and has the risk of side effects such as cell damage caused by radiation exposure. In particular, in the case of pregnant or old patients, MRgFUS treatment involving CT scans is more difficult due to the radiation exposure burden.

### RELATED LITERATURES

(Patent Literature 1) US Patent Publication No. 2011-0235884

### SUMMARY OF THE INVENTION

The present disclosure is designed to solve the above-described problem, and therefore the present disclosure is directed to providing technology that generates a precise computed tomography (CT) image from a magnetic resonance imaging (MRI) image using a trainable artificial neural network model.

The present disclosure is further directed to providing a magnetic resonance-guided ultrasound treatment device capable of achieving precise ultrasound treatment based on skull factor information and acoustic property information acquired by artificial intelligence-based CT imaging technology combined with ultrasound treatment technology without the addition of CT scans.

A method for converting magnetic resonance imaging (MRI) to a computed tomography (CT) image based on artificial intelligence according to an embodiment of the present disclosure is performed by a processor, and includes acquiring training data including an MRI image and a CT image for machine learning; carrying out preprocessing of the training data; training an artificial neural network model using the training data, wherein the artificial neural network model generates a CT image corresponding to the MRI image, and compares the generated CT image with the original CT image included in the training data; receiving an input MRI image to be converted to a CT image; splitting the input MRI image into a plurality of patches; generating patches of a CT image corresponding to the patches of the input MRI image using the trained artificial neural network model; and merging the patches of the CT image to generate an output CT image.

According to an embodiment, training the artificial neural network model may include a first process of generating the CT image corresponding to the MRI image included in the training data using a generator; a second process of acquiring error data by comparing the generated CT image with the original CT image included in the training data using a discriminator; and a third process of training the generator using the error data.

According to an embodiment, the artificial neural network model may be trained to reduce differences between the original CT image and the generated CT image by iteratively performing the first to third processes.

According to an embodiment, the generator may include at least one convolutional layer for receiving input MRI image data and outputting a feature map which emphasizes features of a region of interest; and at least one transposed convolutional layer for generating the CT image corresponding to the input MRI image based on the feature map.

According to an embodiment, the discriminator may include at least one convolutional layer for receiving the input CT image data generated by the generator and outputting a feature map which emphasizes features of a region of interest.

According to an embodiment, the artificial neural network model may generate the CT image corresponding to the MRI image through trained nonlinear mapping.

According to an embodiment, carrying out preprocessing of the training data may include removing an unnecessary area for training by applying a mask to a region of interest in the MRI image and the CT image included in the training data.

There may be provided a computer program stored in a computer-readable recording medium, for performing the method for converting MRI to a CT image based on artificial intelligence according to an embodiment.

A magnetic resonance-guided ultrasound treatment device according to an embodiment of the present disclosure includes an MRI image acquisition unit to acquire an MRI image of a patient; a display unit to display a target tissue for ultrasound treatment on a display based on the MRI image; a CT image generation unit to generate a CT image corresponding to the MRI image using the method for converting MRI to a CT image based on artificial intelligence; a processing unit to acquire factor information and parameter information related to the ultrasound treatment of the target tissue based on the CT image; and an ultrasound output unit to output ultrasound set based on the factor information and the parameter information to the target tissue.

According to an embodiment, the ultrasound output unit may be configured to output high-intensity focused ultrasound to thermally or mechanically remove the target tissue, or low-intensity focused ultrasound to stimulate the target tissue without damage.

A method for converting MRI to a CT image based on artificial intelligence according to another embodiment of the present disclosure is performed by a processor, and includes receiving an input MRI image to be converted to a CT image; splitting the input MRI image into a plurality of patches; generating patches of a CT image corresponding to the patches of the input MRI image using an artificial neural network model trained to generate a CT image corresponding to an arbitrary input MRI image; and merging the patches of the CT image to generate an output CT image.

According to an embodiment, the artificial neural network model may be trained by generating the CT image corresponding to the MRI image included in the input training data, and comparing the generated CT image with the original CT image included in the training data.

According to an embodiment of the present disclosure, it is possible to generate a computed tomography (CT) image corresponding to an input magnetic resonance imaging (MRI) image using an artificial neural network model. According to an embodiment, the artificial neural network model may be trained to minimize differences through competition between a generator which synthesizes a CT image and a discriminator which conducts comparative analysis of the synthesized CT image and the original CT image.

According to an embodiment, acoustic property information of ultrasound may be acquired based on the synthesized CT image, and the information may be used in precise ultrasound treatment. Accordingly, it is possible to reduce the patient's radiation exposure burden and temporal and economical burden caused by the addition of CT scans, and simplify the surgical process of the medical staff.

### BRIEF DESCRIPTION OF THE DRAWINGS

The following is a brief introduction to necessary drawings in the description of the embodiments to describe the technical solutions of the embodiments of the present disclosure or the existing technology more clearly. It should be understood that the accompanying drawings are for the purpose of describing the embodiments of the present disclosure and are not intended to be limiting of the present disclosure. Additionally, for clarity of description, illustration of some elements in the accompanying drawings may be exaggerated and omitted.

FIG. 1 is a flowchart illustrating a method for converting magnetic resonance imaging (MRI) to a computed tomography (CT) image based on artificial intelligence according to an embodiment.

FIG. 2 shows a process of carrying out preprocessing of an MRI image and a CT image included in training data according to an embodiment.

FIG. 3 shows a process of training an artificial neural network model for synthesis of a CT image from an MRI image according to an embodiment.

FIG. 4 shows a process of synthesizing a CT image corresponding to an input MRI image according to an embodiment.

FIGS. 5A-5E show a comparison of quality between a CT image generated according to an embodiment and a real CT image.

FIG. 6 shows a comparison of skull characteristics between a CT image generated according to an embodiment and a real CT image.

FIG. 7 shows the acoustic simulation results of a CT image generated according to an embodiment and a real CT image and differences between them.

FIG. 8 shows a structure of a magnetic resonance-guided ultrasound treatment device using a CT image generated according to an embodiment.

### DETAILED DESCRIPTION OF THE PREFERRED EMBODIMENTS

The following detailed description of the present disclosure is made with reference to the accompanying drawings, in which particular embodiments for practicing the present disclosure are shown for illustrative purposes. These embodiments are described in sufficiently detail for those skilled in the art to practice the present disclosure. It should be understood that various embodiments of the present disclosure are different but do not need to be mutually exclusive. For example, particular shapes, structures and features described herein in connection with one embodiment may be embodied in other embodiment without departing from the spirit and scope of the present disclosure. It should be further understood that changes may be made to the positions or placement of individual elements in each disclosed embodiment without departing from the spirit and scope of the present disclosure. Accordingly, the following detailed description is not intended to be taken in limiting senses, and the scope of the present disclosure, if appropriately described, is only defined by the appended claims along with the full scope of equivalents to which such claims are entitled. In the drawings, similar reference signs denote same or similar functions in many aspects.

The terms as used herein are general terms selected as those being now used as widely as possible in consideration of functions, but they may differ depending on the intention of those skilled in the art or the convention or the emergence of new technology. Additionally, in certain cases, there may be terms arbitrarily selected by the applicant, and in this case, the meaning will be described in the corresponding description part of the specification. Accordingly, it should be noted that the terms as used herein should be interpreted based on the substantial meaning of the terms and the context throughout the specification, rather than simply the name of the terms.

Hereinafter, exemplary embodiments of the present disclosure will be described in detail with reference to the accompanying drawings.

### Method for Converting Magnetic Resonance Imaging (MRI) to a Computed Tomography (CT) Image Based on Artificial Intelligence

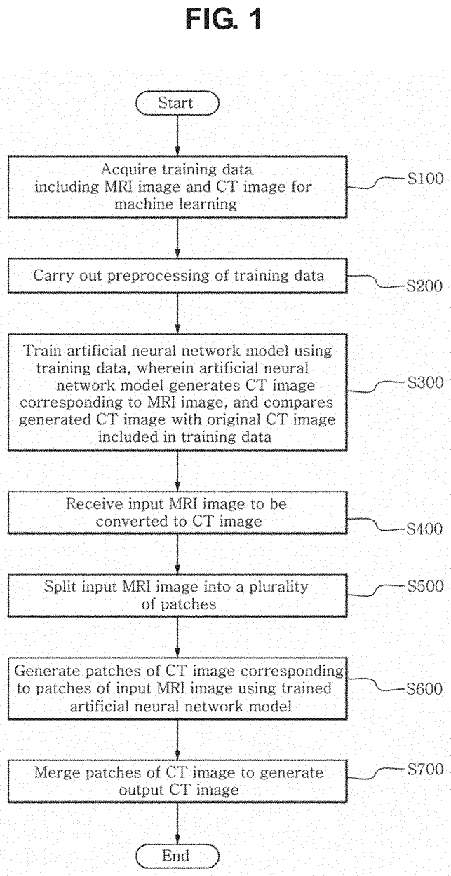

FIG. 1 shows each step of a method for converting MRI to a CT image based on artificial intelligence according to an embodiment.

Referring to FIG. 1, to begin with, the step of acquiring training data including an MRI image and a CT image for machine learning is performed (S100). The machine learning is used for a computer to cluster or classify things or data, and typically includes support vector machine (SVM) and Neural Networks. The present disclosure describes technology that extracts features from an input image (an MRI image) using an artificial neural network model and synthesizes a new corresponding image (a CT image).

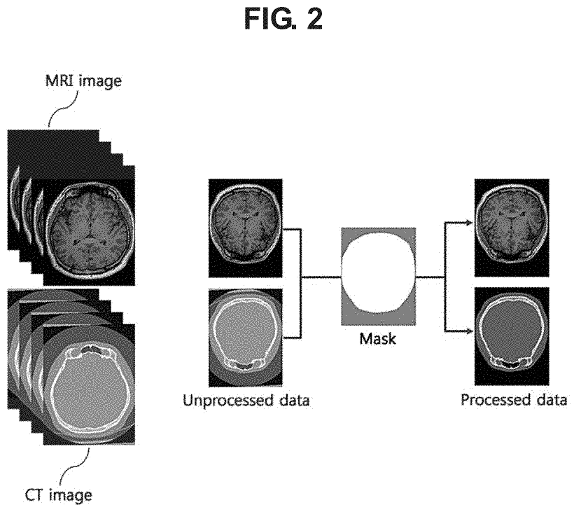

Subsequently, the acquired training data is preprocessed (S200). FIG. 2 shows a process of carrying out preprocessing of the MRI image and the CT image included in the training data. The preprocessing process includes a process of dividing each image into a region of interest and a background and removing the background that is not necessary for training. As shown on the right side of FIG. 2, the brain area and the background image are separated by applying a mask to unprocessed data. Additionally, a process of scaling the intensity of the MRI and the CT image in the range between −1 and 1 may be performed. It is possible to improve the training efficiency through the data preprocessing process.

Subsequently, the step of training the artificial neural network model using the training data is performed (S300). The artificial neural network model is trained to extract features from the input image, generate a CT image corresponding to the input MRI image through trained nonlinear mapping, and generate an image close to the original using error data by comparing the generated CT image with the original CT image.

According to a specific embodiment, the training process of the artificial neural network model includes a first process of generating a CT image corresponding to the MRI image included in the training data using a generator, a second process of acquiring error data by comparing the generated CT image with the original CT image included in the training data using a discriminator, and a third process of training the generator using the error data.

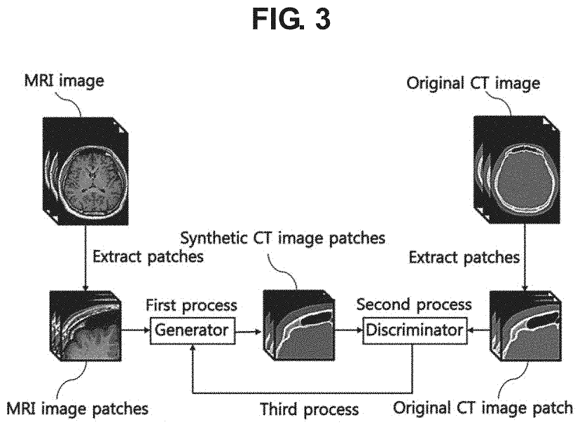

FIG. 3 shows a process of training the artificial neural network model for synthesizing CT image from the MRI image according to an embodiment.

To begin with, the MRI image and the original CT image (i.e., an image actually captured using CT equipment) included in the training data are split into a plurality of 3-dimensional (3D) patches. In case that the original image is used as it is left unsplit, efficiency reduces due to the graphics processing unit (GPU) memory limit, and thus to improve the processing rate and efficiency, after training and generating an image using the split patches, finally the patches are sequentially merged to generate a complete image.

The first process generates synthetic CT image patches corresponding to the MRI image patches included in the training data using the generator. The generator may include multiple layers including at least one convolutional layer and at least one transposed convolutional layer.

According to an embodiment, the convolutional layer that makes up the generator receives input MRI image data and outputs a feature map that emphasizes the features of a region of interest. Specifically, the convolutional layer outputs the feature map that emphasizes the features of an image area by multiplying the input data with filters while the filters move with a predetermined stride. As the image goes through the convolutional layer, the width, height and depth of the image gradually decrease and the number of channels increases. The values of the filters include weight parameters, and the values of the filters are randomly set at the initial step, and in the training step, they are updated for optimization through error backpropagation (updating the weight parameters by propagating the output error of the output layer to the input layer).

The transposed convolutional layer is a layer that learns a process of synthesizing the feature maps extracted by the convolutional layer into a target output image and restoring the size (upsampling). The transposed convolutional layer outputs the feature maps by multiplying input data with filters while the filters move with a predetermined stride, and transposes the input/output size as opposed to the convolutional layer. That is to say, as the image goes through the transposed convolutional layer, the width, height and depth of the image gradually increases, and the number of channels decreases. A new image is generated based on the extracted features by transposing the convolutional layer.

According to an embodiment, the convolutional layer or the transposed convolutional layer of the generator may be used together with instance normalization for normalizing the data distribution of the feature maps and an activation function for determining the range of each output value. The instance normalization serves to prevent overfitting in which the filter values (weights) of convolution or transposed convolution fit training data well and perform worse on test data in the training process, thereby stabilizing the training process. The feature maps are normalized through the mean and the standard deviation (for each instance fed into the model) to stabilize the data distribution of the feature maps. After training, the actual input test data is also equally normalized through the mean and the standard deviation stored during the training process, thereby generating an output image from data having a different distribution from the training data more stably.

When combined with the convolutional layer or the transposed convolutional layer, the activation function determines the range of the output value that will be passed from each layer to other layer and sets a threshold value for deciding which values will be passed. Additionally, nonlinearity is added to a deep learning model, and by the addition of the nonlinearity, the layers of the deep learning model become deeper, and derivative values are very small and converge to 0, thereby reducing the Gradient vanishing effect that the weight parameters are not updated. The activation function may include, for example, ReLu activation function that when input data is equal to or smaller than 0, outputs 0, and when input data is larger than 0, preserves the value, LeakyReLu activation function which plays a similar role to the ReLu activation function, but when the input value is smaller than 0, multiplies the input value by 0.1 to have a non-zero output, and when the input value is larger than 0, preserves the value, Tanh activation function that causes input data to have a value between −1 and 1, and Sigmoid activation function that causes input data to have a value between 0 and 1.

The second process acquires error data by comparing the generated CT image with the original CT image included in the training data using the discriminator. The discriminator may include continuous convolutional layers, and as opposed to the generator, each convolutional layer is configured to receive input CT image data and output a feature map that emphasizes features of a region of interest. In the same way as the generator, each convolutional layer may be used together with instance normalization for normalization of the data distribution of the feature map and an activation function for determining the range of each output value.

According to an embodiment, the generator may include at least one residual block layer, and since the deeper layers of the model make optimization more difficult, the residual block layer serves to facilitate the model training. The residual block layer is repeated between the convolutional layer (Encoder) which reduces the width and height of the image and widens channelwise, and the transposed convolutional layer (Decoder) which restores the width and height of the image and the channels into its original dimensions. One residual block includes convolution-instance normalization-ReLu activation-convolution-instance normalization, and here, the convolution outputs an image having the same width, height, depth and channel size as the input image through adjustment of the filters and the stride values. That is, it is aimed at passing the input data to the next layer with the minimized information loss, but not extracting or restoring the features of the input data. For example, the residual block is set in such a way that the input x value is added to the output of the residual block, which induces the learning of a difference F(x) between input x and output H(x), not the output H(x) of the input data x. Accordingly, the previously learned input data x is taken as it stands and added to the output, so that only residual information F(x) is learned, thereby simplifying the model training process.

The third process trains the generator using the error data between the synthetic CT image and the original CT image. That is, the CT image synthesized through the MRI image may be compared with the original CT image actually captured through CT equipment, and the comparison results may be returned to the generator to improve the performance of the generator so as to output subsequent results that are more similar to the original CT image. The artificial neural network model according to an embodiment may be trained to reduce differences between the original CT image and the generated CT image by iteratively performing the first to third processes through various training data.

As described above, the generator may generate an image through nonlinear mapping, and the discriminator may generate a more precise image (i.e., an image close to the original) with the increasing training iterations through a Generative Adversarial Network (GAN) model that classifies the generated image and the original image.

Referring back to FIG. 1, after the training of the artificial neural network model, the step of receiving input MRI image data to be converted to a CT image (S400) and the step of splitting the input MRI image into a plurality of patches (S500) is performed. The input MRI image is an image of the patient's brain or body part on which surgery will be actually performed, captured through MRI equipment. As described above, it is possible to overcome the GPU memory limit by splitting the MRI image into a plurality of 3D patches and generating a corresponding CT image patch for each patch.

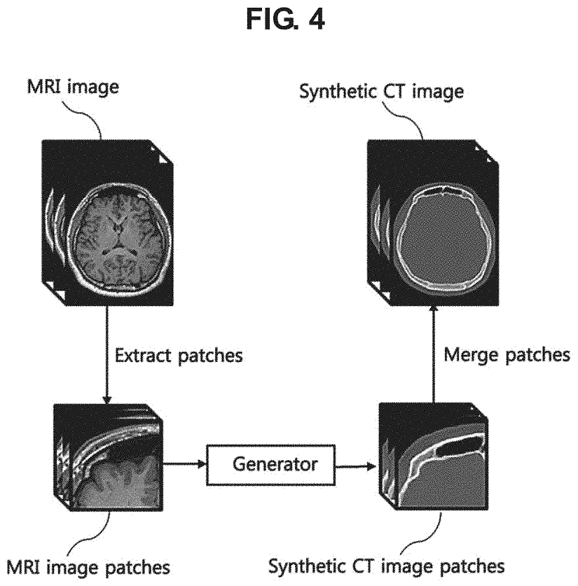

Subsequently, the step of generating patches of a CT image corresponding to the patches of the input MRI image using the trained artificial neural network model is performed (S600). As described above, the generator generates each CT image patch corresponding to each MRI image patch through nonlinear mapping, and sequentially merges the patches to generate a CT image. FIG. 4 shows a process of generating the synthetic CT image from the input MRI image.

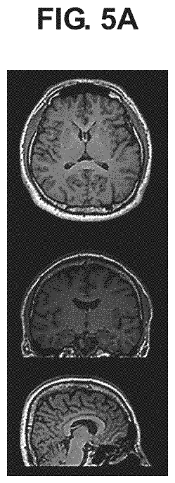

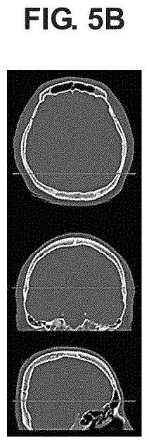

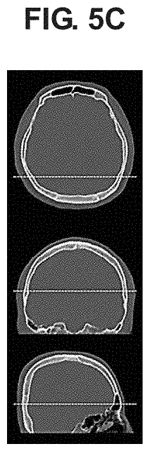

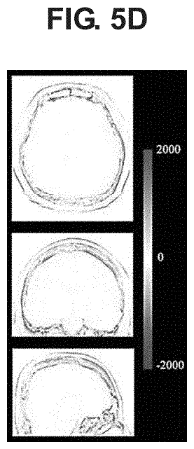

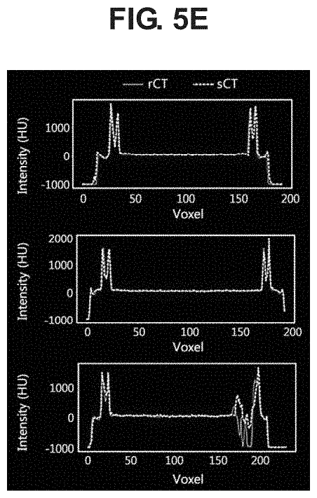

FIGS. 5A-5E show a photographic image and a graph showing a comparison of quality between the CT image generated according to an embodiment and the real CT image. FIG. 5A shows the MRI image of the brain, FIG. 5B shows the real CT image of the brain, and FIG. 5C shows the synthetic CT image generated using the artificial neural network model. FIG. 5D shows a difference of the synthetic CT image (sCT) generated according to an embodiment and the real CT image (rCT), and FIG. 5E is a graph showing intensity as a function of voxel (graphical information of an edge defining a point in a 3D space) of each image. As can be seen from FIG. 5D to FIG. 5E, the cross-section of the synthetic CT image and the cross-section of the real CT image almost match each other, and the intensity is also similarly measured.

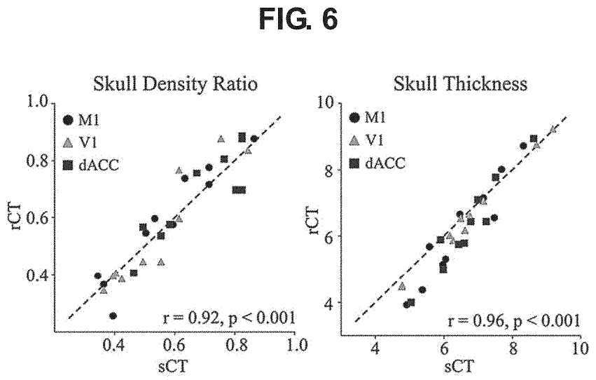

FIG. 6 shows a result of comparing the skull characteristics in a specific brain area between the generated CT image (sCT) according to an embodiment and the real CT image (rCT). As shown, it can be seen that the Pearson's correlation coefficient (p<0.001) is 0.92 and the skull thickness is 0.96, showing high similarity of the skull density ratio over the entire area. This signifies that in addition to the acoustic properties, it is possible to induce the similar simulation results to the real CT.

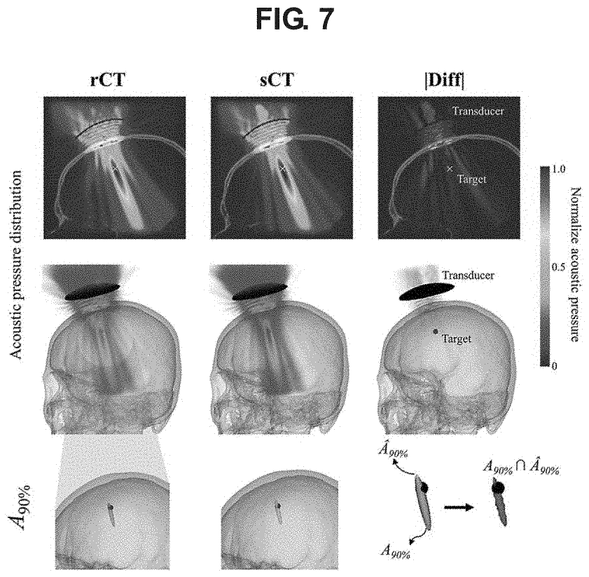

FIG. 7 shows 2D and 3D representations of the difference in acoustic simulation results (Diff) of the generated CT image (sCT) according to an embodiment and the real CT image (rCT). As shown, it can be seen that an acoustic pressure difference dACC of the target position almost matches, and areas in which ultrasound converges onto the focus point also almost overlap. The following table shows the maximum acoustic pressure and focal distance error calculated by applying the real CT image and the synthetic CT image. Simulation was performed on 10 subjects in the subcortical region (M1; primary motor cortex, V1; primary motor cortex, dACC; dorsal anterior cingulate cortex).

TABLE 1

| Target | Maximum acoustic | Focaldice | Focal distance |
|---|---|---|---|
| position | pressure (%) | coefficient (%) | error (mm) |
| M1 | 3.72 ± 2.68 | 0.81 ± 0.08 | 1.09 ± 0.59 |
| V1 | 2.11 ± 1.65 | 0.89 ± 0.06 | 0.76 ± 0.48 |
| dACC | 4.87 ± 3.28 | 0.84 ± 0.07 | 0.95 ± 0.63 |
| Mean | 3.57 ± 2.86 | 0.85 ± 0.07 | 0.93 ± 0.59 |

As shown in the above table, the maximum acoustic pressure exhibits an error ratio of 4% or less on average all over the targets. This signifies that the synthesized CT image rather than the real CT image is suitable for use in ultrasound treatment.

The method for converting MRI to a CT image based on artificial intelligence according to an embodiment may be implemented as an application or in the format of program instructions that may be executed through a variety of computer components and recorded in computer readable recording media. The computer readable recording media may include program instructions, data files and data structures alone or in combination.

Examples of the computer readable recording media include hardware devices specially designed to store and execute the program instructions, for example, magnetic media such as hard disk, floppy disk and magnetic tape, optical media such as CD-ROM and DVD, magneto-optical media such as floptical disk, and ROM, RAM and flash memory.

Examples of the program instructions include machine code generated by a compiler as well as high-level language code that can be executed by a computer using an interpreter. The hardware device may be configured to act as one or more software modules to perform the processing according to the present disclosure, and vice versa.

According to the above embodiments, it is possible to generate the CT image corresponding to the input MRI image using the artificial neural network model. The artificial neural network model can improve the performance through adversarial training of the generator and the discriminator, and generate the precise CT image having the error ratio of 10% or less.

### Magnetic Resonance-Guided Focused Ultrasound (MRgFUS) Treatment Device

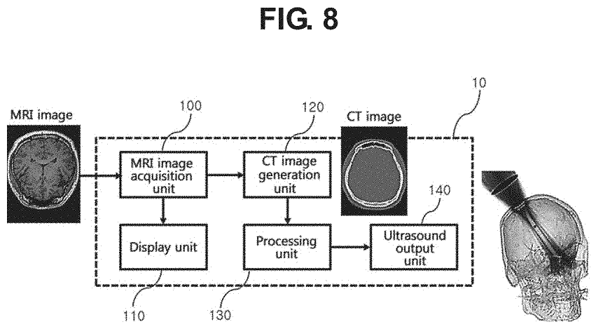

FIG. 8 shows the structure of the magnetic resonance-guided ultrasound treatment device according to an embodiment using the CT image generated by the above-described method.

Referring to FIG. 8, the ultrasound treatment device 10 includes an MRI image acquisition unit 100 to acquire a patient's MRI image, a display unit 110 to display a target tissue for ultrasound treatment on a display based on the MRI image, a CT image generation unit 120 to generate a CT image corresponding to the MRI image using the method for converting MRI to a CT image based on artificial intelligence, a processing unit 130 to acquire factor information and parameter information related to the ultrasound treatment of the target tissue based on the CT image, and an ultrasound output unit 140 to output ultrasound set based on the factor information and the parameter information to the target tissue.

The MRI image acquisition unit 100 receives the input MRI image and carries out preprocessing. The preprocessing process may include dividing the image into a region of interest and a background through a mask and removing the background.

The display unit 110 displays the MRI image on the display to allow a surgeon to perform ultrasound treatment while observing the target tissue.

The CT image generation unit 120 generates the corresponding CT image from the input MRI image using the method for converting MRI to a CT image based on artificial intelligence. As described above, the artificial neural network model improves the precision of the synthetic CT image through adversarial training of the generator and the discriminator (that is, trained to minimize differences between the synthetic CT image and the real CT image).

The processing unit 130 acquires skull factor information or acoustic property information necessary for the ultrasound treatment based on the generated CT image. Since the direction of travel of the focused ultrasound, the focus point and the pressure at the focus point may vary depending on the thickness, location and shape of the skull through which ultrasound penetrates, it is necessary to pre-acquire such information through the CT image for the purpose of precise treatment.

Finally, the ultrasound output unit 140 outputs ultrasound set based on the information (skull factor information, ultrasound parameters, acoustic property information, etc.) identified in the generated CT image. The ultrasound output unit 140 includes a single ultrasonic transducer or a series of ultrasonic transducers to convert alternating current energy to mechanical vibration, and generates and outputs ultrasound according to the set value such as acoustic pressure, waveform and frequency. The output ultrasound overlaps to form an ultrasound beam which in turn converges at a target focus point to remove or stimulate the target tissue. According to an embodiment, the ultrasound output unit 140 is configured to output high-intensity focused ultrasound to thermally or mechanically remove the target tissue or low-intensity focused ultrasound to stimulate the target tissue without damage.

According to the configuration of the ultrasound treatment device described above, it is possible to achieve precise ultrasound treatment using the acoustic property information acquired from the synthesized CT image without the addition of CT scans. Accordingly, it is possible to reduce the patient's radiation exposure burden and temporal and economical burden caused by the addition of CT scans, and simplify the surgery process of the medical staff.

While the present disclosure has been hereinabove described with reference to the embodiments, it will be apparent to those having ordinary skill in the corresponding technical field that various modifications and changes may be made to the present disclosure without departing from the spirit and scope of the present disclosure defined in the appended claims.

What is claimed is:

1. A method for converting magnetic resonance imaging (MRI) to a computed tomography (CT) image based on artificial intelligence, performed by a processor, the method comprising:
acquiring training data including an MRI image and a CT image for machine learning;
carrying out preprocessing of the training data;
training an artificial neural network model using the training data, wherein the artificial neural network model generates a CT image corresponding to the MRI image, and compares the generated CT image with the original CT image included in the training data;
receiving an input MRI image to be converted to a CT image;
splitting the input MRI image into a plurality of patches;
generating patches of a CT image corresponding to the patches of the input MRI image using the trained artificial neural network model; and
merging the patches of the CT image to generate an output CT image.

2. The method for converting MRI to a CT image based on artificial intelligence according to claim 1, wherein training the artificial neural network model comprises:
a first process of generating the CT image corresponding to the MRI image included in the training data using a generator;
a second process of acquiring error data by comparing the generated CT image with the original CT image included in the training data using a discriminator; and
a third process of training the generator using the error data.

3. The method for converting MRI to a CT image based on artificial intelligence according to claim 2, wherein the artificial neural network model is trained to reduce differences between the original CT image and the generated CT image by iteratively performing the first to third processes.

4. The method for converting MRI to a CT image based on artificial intelligence according to claim 2, wherein the generator includes:
at least one convolutional layer for receiving input MRI image data and outputting a feature map which emphasizes features of a region of interest; and
at least one transposed convolutional layer for generating the CT image corresponding to the input MRI image based on the feature map.

5. The method for converting MRI to a CT image based on artificial intelligence according to claim 2, wherein the discriminator includes at least one convolutional layer for receiving the input CT image data generated by the generator and outputting a feature map which emphasizes features of a region of interest.

6. The method for converting MRI to a CT image based on artificial intelligence according to claim 1, wherein the artificial neural network model generates the CT image corresponding to the MRI image through trained nonlinear mapping.

7. The method for converting MRI to a CT image based on artificial intelligence according to claim 1, wherein carrying out preprocessing of the training data comprises:
removing an unnecessary area for training by applying a mask to a region of interest in the MRI image and the CT image included in the training data.

8. A computer program stored in a computer-readable recording medium, for performing the method for converting magnetic resonance imaging (MRI) to a computed tomography (CT) image based on artificial intelligence according to claim 1.

9. A magnetic resonance-guided ultrasound treatment device, comprising:
a magnetic resonance imaging (MRI) image acquisition unit to acquire an MRI image of a patient;
a display unit to display a target tissue for ultrasound treatment on a display based on the MRI image;
a computed tomography (CT) image generation unit to generate a CT image corresponding to the MRI image using the method for converting MRI to a CT image based on artificial intelligence according to claim 1;
a processing unit to acquire factor information and parameter information related to the ultrasound treatment of the target tissue based on the CT image; and
an ultrasound output unit to output ultrasound set based on the factor information and the parameter information to the target tissue.

10. The magnetic resonance-guided ultrasound treatment device according to claim 9, wherein the ultrasound output unit is configured to output high-intensity focused ultrasound to thermally or mechanically remove the target tissue, or low-intensity focused ultrasound to stimulate the target tissue without damage.

11. A method for converting magnetic resonance imaging (MRI) to a computed tomography (CT) image based on artificial intelligence, performed by a processor, the method comprising:
receiving an input MRI image to be converted to a CT image;
splitting the input MRI image into a plurality of patches;
generating patches of a CT image corresponding to the patches of the input MRI image using an artificial neural network model trained to generate a CT image corresponding to an arbitrary input MRI image; and
merging the patches of the CT image to generate an output CT image.

12. The method for converting MRI to a CT image based on artificial intelligence according to claim 11, wherein the artificial neural network model is trained by generating the CT image corresponding to the MRI image included in the input training data, and comparing the generated CT image with the original CT image included in the training data.

13. A magnetic resonance-guided ultrasound treatment device, comprising:
a magnetic resonance imaging (MRI) image acquisition unit to acquire an MRI image of a patient;
a display unit to display a target tissue for ultrasound treatment on a display based on the MRI image;
a computed tomography (CT) image generation unit to generate a CT image corresponding to the MRI image using the method for converting MRI to a CT image based on artificial intelligence according to claim 11;
a processing unit to acquire factor information and parameter information related to the ultrasound treatment of the target tissue based on the CT image; and
an ultrasound output unit to output ultrasound set based on the factor
information and the parameter information to the target tissue.
